(19) United States
(12) Patent Application Publication
(10) Pub. No.: US 2012/0178757 A1
(43) Pub. Date: Jul. 12, 2012

(54) Method For The Prevention Of Cancer Metastasis

(75) Inventor: Desmond George Powe, Nottingham (GB)

(73) Assignee: NOTTINGHAM UNIVERSITY HOSPITALS NHS TRUST, Nottingham (GB)

(21) Appl. No.: 13/314,437

(22) Filed: Dec. 8, 2011

Related U.S. Application Data
(60) Provisional application No. 61/421,405, filed on Dec. 9, 2010.

(51) Int. Cl. A61K 31/5377; C40B 30/04; G01N 33/567; C12Q 1/68; A61K 31/16; A61K 31/135; A61K 31/165; A61K 31/405; A61K 31/47; A61K 31/403; A61K 31/155; A61K 31/352; A61K 31/18; A61K 31/517; A61K 31/495; A61K 31/454; A61K 31/4164; A61P 35/04; G01N 33/566

(52) U.S. Cl. 514/236.2; 435/7.92; 506/9; 435/7.21; 435/6.12; 514/630; 514/651; 514/620; 514/652; 514/415; 514/312; 514/411; 514/634; 514/456; 514/605; 514/266.24; 514/252.17; 514/250; 514/323; 514/603; 514/401

(57) ABSTRACT

A method is provided for the prevention of cancer metastasis in a patient comprising administering an adrenergic receptor antagonist to a patient in need thereof. The invention also includes an adrenergic receptor antagonist for use in the prevention of tumour recurrence in a patient. Also provided is a kit of parts for use in such methods comprising an adrenergic receptor antagonist, and an administration vehicle. A method is further included for identifying a patient at risk of developing cancer comprising the step of assaying for levels of expression of adrenergic receptor genes and proteins in said patient.

The present invention relates to methods for prevention of cancer metastasis in patients.

Although an estimated 207,090 patients are diagnosed with breast cancer in the US each year death rates are declining in part due to adjuvant therapies including the use of ER-antagonists and anti-HER2 (trastuzumab/Herceptin®) therapy. Nonetheless, approximately 30% of treated breast cancer patients develop distant metastases and these significantly account for 90% of breast cancer deaths. Consequently, therapeutic strategies are needed that target metastasis.

Although a heterogeneous disease, breast cancer can broadly be classified into 4 main molecular types including luminal-like (oestrogen receptor (ER) positive), basal-like, HER2 positive, and normal-like breast groups. Targeted therapeutic antagonists can be used to inhibit ER and HER2 proteins giving improved prognosis in the luminal and HER2 positive patient groups, respectively. Nonetheless, approximately 30% of all breast patients show metastasis formation which negatively impacts on survival, so much so that survival time can be considered in two steps: the initial metastasis-free interval and secondly, the time after metastasis development to death (Engel et al. Eur J Cancer 2003, 39(12):1794-1806).

Metastasis formation involves cancer cell migration from the primary tumour via lympho-/hematogenic routes and is tightly regulated by exogenous signal molecules, including ligands to G protein-coupled receptors (GPCRs), namely chemokines and neurotransmitters (Entschladen et al Lancet Oncol 2004, 5(4):254-258).

In previous in vitro cell migration studies it has been shown that the stress catecholamine hormone norepinephrine is a potent inducer of migratory activity in carcinoma cell lines of colon (Masur et al. Cancer Res 2001; 61(7):2866-9), prostate (Lang et al. Int J Cancer 2004; 112(2):231-8), ovary (Sood et al. Clin Cancer Res 2006; 12(2):369-75) and breast (Drell et al Breast Cancer Research and Treatment 2003; 80(1):63-70) tissue origin, and this finding has been confirmed in a mouse model (Palm et al. International Journal of Cancer 2006; 118(11):2744-2749).

These earlier reports indicated that cell migration is mediated by adrenergic receptors (adrenoceptors) and that the process can be inhibited by the beta-blocker adrenoceptor antagonist drug propranolol which is non-selective for β1AR and β2AR.

Adrenoceptor antagonists comprise two main groups, alpha- and beta-receptor specific antagonists (Dorn G W & Liggett S B Cts-Clinical and Translational Science 2008, 1(3):255-262; Rosini et al. Current Topics in Medicinal Chemistry 2007, 7(2):147-162), and have recently received attention for offering a novel therapeutic approach in the treatment of cancer. One such study showed that coincidental treatment with α1-adrenoceptor antagonists (doxazosin and terazosin) for hypertension or benign prostate hyperplasia significantly lowered the incidence of prostate cancer (Harris et al. Journal of Urology 2007, 178(5):2176-2180). These therapeutic benefits appear to extend to several cancer types (Algazi et al Letters in Drug Design & Discovery 2006, 3(9):653-661).

Other recent studies have shown that propranolol can reduce proliferation in human pancreatic cell lines (Al-Wadei et al Anti-Cancer Drugs 2009; 20(6):477-482; Zhang et al Pancreas 2009; 38(1):94-100). Beta-blockers are a diverse range of drugs with pharmacological properties which are commonly used in the treatment of heart disease and hypertension as they are effective at reducing the heart rate and lowering blood pressure. Beta-blockers can be categorized into cardioselective (specificity for beta-adrenoceptors) and non-selective (inhibit alpha- and beta-adrenoceptors) types.

Translation of laboratory adrenoceptor/antagonist models into a clinical setting consist of only a few recent observational epidemiological studies investigating their effect on the incidence of cancer and showing that beta-blocker drugs reduce the incidence of multiple cancer types (Algazi et al. Letters in Drug Design & Discovery 2006, 3(9):653-661). Similarly, alpha antagonists also appear to limit cancer growth as demonstrated by Hui et al (Eur J Cancer 2008, 44(1):160-166). The selective alpha(1) adrenoceptor antagonist doxazosin was found to inhibit proliferation and induce apoptosis in in vitro breast cancer cells. Beta-blockers can be safely taken for long periods without any adverse carcinogenic risks (Assimes T et al. Pharmacoepidemiol Drug Saf, 2008, 17:1039-1049). However, a possible limitation in the novel application of some adrenoceptor antagonists in cancer treatment is their side effects notably caused by smooth muscle relaxation, resulting in cardiovascular-associated hypotension (Lowe F C, Clinical Therapeutics 2004, 26(11):1701-1713). In the case of alpha antagonists, new tissue specific versions are being engineered to have fewer side effects (Rosini et al Current Topics in Medicinal Chemistry 2007, 7(2):147-162); Chiu et al. Expert Opinion on Therapeutic Patents 2008, 18(12):1351-1360).

However, these initial studies are in vitro experiments conducted on cancer cell lines in culture or only relate to the incidence of cancer in cohorts of human patients. Laboratory-based cancer cell lines do not equate with actual cases of clinical disease in patients. Furthermore, the incidence of cancer is also not predictive of metastasis formation.

In particular, for the development of new therapies for the treatment or prevention of cancer in a patient, the behaviour of isolated cells in culture is not predictive of the successful outcome of a clinically relevant therapy in patients.

Beta-blockers have been shown to reduce the incidence of endocrine-regulated prostate cancer (Fitzpatrick et al. Annals of Epidemiology 2001; 11(8):534-542), but this beneficial effect is not just limited to inhibition of beta receptors because patients receiving alpha antagonists also show a reduced incidence of prostate (Harris et al. Journal of Urology 2007; 178(5):2176-2180) and bladder (Martin et al Gene Therapy and Molecular Biology 2008; 12B:253-257) cancer. Moreover, a generalised reduction in the incidence of all cancer types has been reported in beta-blocker treated patients (Algazi et al Letters in Drug Design & Discovery 2006; 3(9):653-661).

Tumour metastasis is a complex process and contributes to the majority of deaths seen in breast cancer disease. New and effective therapeutic strategies targeted at metastasis are needed, given the current poor survival rates for some types of cancer. Metastases are difficult to treat and current therapies including chemotherapy and radiotherapy are not always effective, are expensive, involve specialist management, and may have harmful side effects.

However, the present inventors have discovered that beta-blockers can be used to prevent cancer metastasis in patients which represents a significant step forward in providing an effective clinical therapy for such diseases.

Beta-blocker drugs along with ACE-inhibitors, calcium channel antagonists, imidazoline receptor antagonists, and diuretics are clinically well characterized for the therapeutic treatment of hypertension and are proven in reducing life-threatening cerebrovascular events. However, such drugs have not previously been described or shown to reduce mortality from cancer. The discovery of a previously unknown clinically relevant therapeutic indication for this class of drugs is therefore particularly useful and advantageous.

As discussed above, earlier studies by Algazi et al (2006) investigated the effect of beta-blockers on the incidence of cancer (in other words the risk of cancer developing in a patient) and found that there is reduced risk in patients treated with beta-blockers. However, there was no disclosure or suggestion of any reduction in mortality from such prophylactic treatment. It is therefore important to understand that the results of Algazi et al (2006) do not teach any reduced mortality as is seen from the present invention. In addition, the present inventors also report reduced tumour recurrence and metastases which is not disclosed or suggested previously.

In addition, the present inventors have discovered that levels of expression of alpha- and beta adrenergic receptor (adrenoceptor) proteins are increased in (breast) cancer and have a direct correlation with clinical outcome including patient survival. The inventors propose that determining tissue adrenoceptor levels can be used to screen for patients which would benefit from targeted prophylactic therapy with adrenoceptor antagonists. The therapy may use non-selective beta-blockers (alpha- and beta-activity) as well as selective alpha- or beta-blockers.

According to a first aspect of the invention there is provided a method for the prevention of cancer metastasis in a patient comprising administering an adrenergic receptor antagonist to a patient in need thereof.

According to the National Cancer Institute, “metastasis” is defined as the spread of cancer from one part of the body to another. A tumour formed by cells that have spread is called a “metastatic tumour” or a “metastasis.” The metastatic tumour contains cells that are like those in the original (primary) tumour. The present invention provides a means to treat and/or prevent such metastatic cancer tumours from developing.

Methods in accordance with the present invention may be used to prevent cancer metastasis. Particularly suitable forms of cancer metastasis which can be treated according to the invention are metastasis associated with breast cancer, colon cancer, prostate cancer or ovarian cancer.

The methods of the present invention may be of specific use in preventing metastasis in patients suffering from basal-like breast cancer. This form of breast cancer is an aggressive form of disease and results in currently high levels of mortality. Some forms of basal type breast cancer may be characterised by mutations in the BRCA1 and BRCA2 gene. The present invention may be particularly suitable in reducing or preventing metastases in patients with basal-type cancer (including BRCA1/BRCA2 mutation positives). A key characteristic of basal-type cancers is their propensity to spread (metastasise) and a failure to respond to conventional treatments.

Due to previous in vitro cell line studies and sterol and catecholamine hormonal regulatory signalling similarities, it is highly probable that this invention applies to prostate cancer. The incidence of basal-type and prostate cancer is increased along racial lines. African populations show an increased frequency of these diseases and the inventors propose that this may be due to the increased frequency of adrenoceptor gene mutations that have been reported (Chronic heart failure: Beta-blockers and pharmacogenetics. J Azuma & S Nonen. Eur. J. Clin. Pharmacol. (2009). 65:3-17). Individuals carrying these gene mutations may be targeted for adrenoceptor antagonist therapy.

The methods of the present invention may be of specific use in preventing tumour recurrence. For example, if excision of the primary tumour is incomplete, the tumour may recur. There are instances (e.g. diffuse lobular breast cancer) when complete surgical removal can be difficult. Consequently, the methods of the present invention provide a useful therapeutic advance in treating such disease recurrence.

The adrenergic receptor antagonist used in accordance with the methods of the present invention may be administered by any suitable means.

This aspect of the invention therefore extends to an adrenergic receptor antagonist for use in the prevention of cancer metastasis. The invention also includes the use of an adrenergic receptor antagonist in the manufacture of a medicament for use in the prevention of cancer metastasis.

As used herein, the term “treatment” includes any regime that can benefit a human or a non-human animal. The treatment is a prophylactic (preventive) treatment.

The adrenergic receptor antagonists for use in accordance with the methods of the present invention may be employed in combination with the pharmaceutically acceptable carrier or carriers. Such carriers may include, but are not limited to, saline, buffered saline, dextrose, liposomes, water, glycerol, polyethylene glycol, ethanol and combinations thereof. Pharmaceutical compositions comprising adrenergic receptor antagonists may therefore be formulated as required according to standard procedures in the art.

The pharmaceutical compositions may be administered in any effective, convenient manner effective for treating a patient's disease including, for instance, administration by oral, topical, intravenous, intramuscular, intranasal, or intradermal routes among others. In therapy or as a prophylactic, the active agent may be administered to an individual as an injectable composition, for example as a sterile aqueous dispersion, preferably isotonic.

For administration to mammals, and particularly humans, it is expected that the daily dosage of the active agent will be from 0.01 mg/kg body weight, typically around 1 mg/kg. The physician in any event will determine the actual dosage which will be most suitable for an individual and which will be dependent on factors including the age, weight, sex and response of the individual. The above dosages are exemplary of the average case. There can, of course, be instances where higher or lower dosages are merited, and such are within the scope of this invention.

An adrenergic receptor antagonist is a substance that acts to inhibit the action of adrenergic receptors. Such pharmaceutically active agents can therefore be classed as a type of sympatholytic. Adrenergic receptor antagonist can also be divided into alpha-blockers and beta-blockers according to the class of adrenergic receptor which is acted upon by the substance. In some instances the beta-blocker may be active against the beta-1 receptor (beta-1 selective), while in other situations the beta-blocker may have non-selective activity against the beta-1 and beta-2 receptors.

The adrenergic receptor antagonist may be an alpha-adrenergic receptor antagonist or a beta-adrenergic receptor antagonist.

The adrenergic receptor antagonist may be administered with another pharmaceutically active agent, for example another agent active in the treatment of cancer, or an agent useful in reducing or ameliorating side effects of the adrenergic receptor antagonist. The therapy may comprise the separate, subsequent, or simultaneous administration of the active agents according to the present invention.

It is generally recommended that beta-blockers are titred under close medical supervision. Starting doses tend to be in the range of 2.5-25 mg/day, increasing up to 200 mg/day, dependent on a number of factors including the type of beta-blocker. Exemplary dosage regimes may be once or twice daily of dosages of 20 mg, 30 mg, 40 mg, 50 mg, 60 mg, 70 mg, 80 mg or 90 mg. For example a dosage may be 80 mg twice daily, or a single dosage of 50 mg once daily.

Where the method comprises the use of a beta-adrenergic receptor antagonist, it may be co-administered with another different beta-adrenergic receptor antagonist, or an alpha-adrenergic receptor antagonist.

The group of beta-adrenergic receptor antagonists which may be used in the present invention include, but are not limited to, Acebutolol, Alprenolol, Atenolol, Bisoprolol, Bucinodolol, Carteolol, Carvedilol, Celiprolol, Labetalol, Metoprolol, Nadolol, Nebivolol, Oxyprenolol, Penbutalol, Pindolol, Propranolol, Sotalol, Timolol

Carvedilol and Labetalol have both alpha-blocker and beta-blocker activities.

The group of alpha-adrenergic receptor antagonists which may be used in the present invention include, but are not limited to, Alfuzosin, Doxazosin, Ergotamine, Indoramin, Prazosin, Phenoxybenzamine, Phentolamine, Tamsulosin, Terazosin, Tolazoline, Trimazosin

The adrenergic receptor antagonist of the invention may be employed alone or in conjunction with other compounds, such as therapeutic compounds, e.g. anti-inflammatory drugs, cytotoxic agents, cytostatic agents and/or antibiotics. The adrenergic receptor antagonists useful in the present invention are preferably provided in an isolated form, and preferably are purified to homogeneity. The components may be prepared in the form of a kit which may comprise instructions as appropriate.

A kit of parts for use in a method of the present invention can be prepared comprising an adrenergic receptor antagonist, and an administration vehicle including, but not limited to, tablets for oral administration, inhalers for lung administration and injectable solutions for intravenous administration.

According to a second aspect of the invention there is provided a method for identifying a patient at risk of developing cancer comprising the step of assaying for levels of expression of adrenergic receptor proteins in said patient.

The adrenergic receptor protein may be an alpha adrenergic receptor protein or a beta adrenergic receptor protein. For example, the alpha adrenergic receptor (alpha adrenoceptor) may be the alpha α1b or α2c receptor protein. The beta adrenergic receptor (beta adrenoceptor) may be the β₂ adrenoceptors.

The expression of the alpha adrenergic receptor protein or a beta adrenergic receptor protein can be suitably assayed in malignant tissue. For example, the step of assaying for receptor expression may use immunohistochemistry, ELISA, microarray/DNA chip assays and/or PCR-based nucleic acid (DNA or RNA) amplification techniques.

Associations between adrenoceptor expression and clinical outcome, and other tumour-relevant biological markers can be statistically assessed subsequently after assays for the levels of receptor expression are made.

A significant advantage of the claimed method of this aspect of the invention is that it permits the identification of patients who would benefit from early intervention in terms of prophylactic anti-cancer therapy, including the prophylactic administration of alpha- and/or beta-blockers.

All preferred features of the second and subsequent aspects of the invention are as for the first aspect mutatis mutandis.

The present invention will now be described by way of reference to the following examples which are provided for the purposes of illustration only and are not to be construed as being limiting on the invention.

Reference in the Examples is also made to a number of drawings in which:

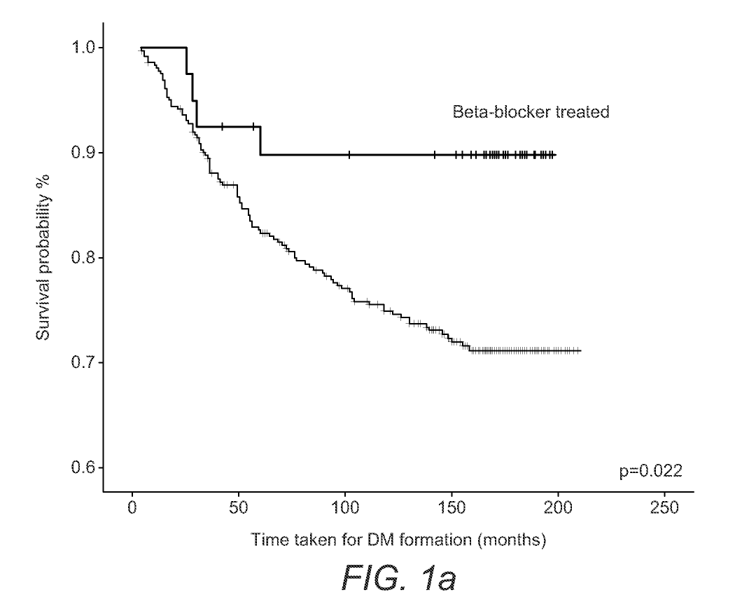

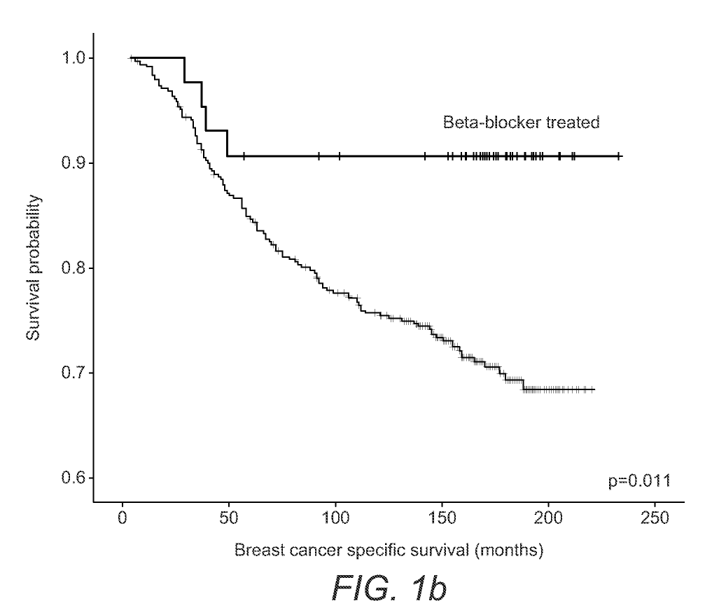

FIG. 1a shows hypertensive BC patients therapeutically treated with beta-blockers that showed significantly (p=0.022) longer times before acquiring metastases compared to non-treated patients.

FIG. 1b shows hypertensive BC patients receiving beta-blocker therapy that showed significantly (p=0.011) improved 10 year survival rates compared to non-treated patients.

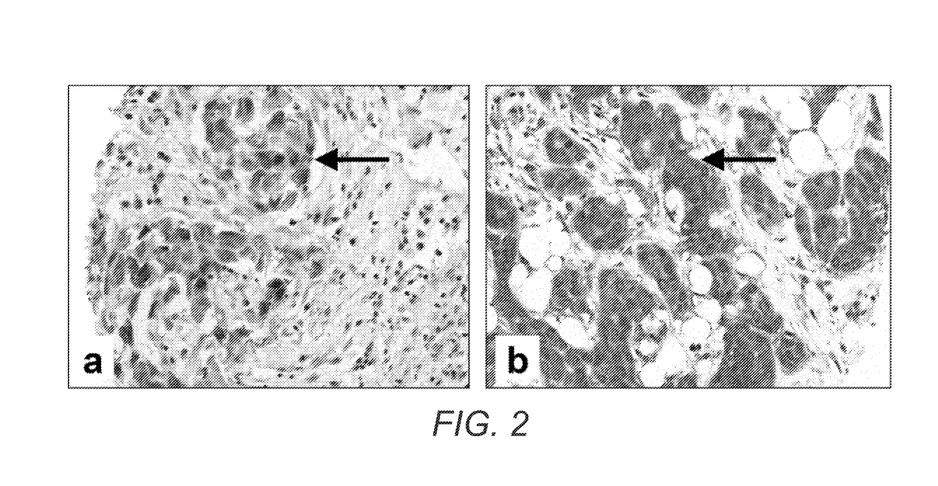

FIG. 2 shows immunohistochemistry used to localise antigens for alpha1b (a), alpha2c (b) and beta2 adrenoceptor proteins in breast cancer (arrow).

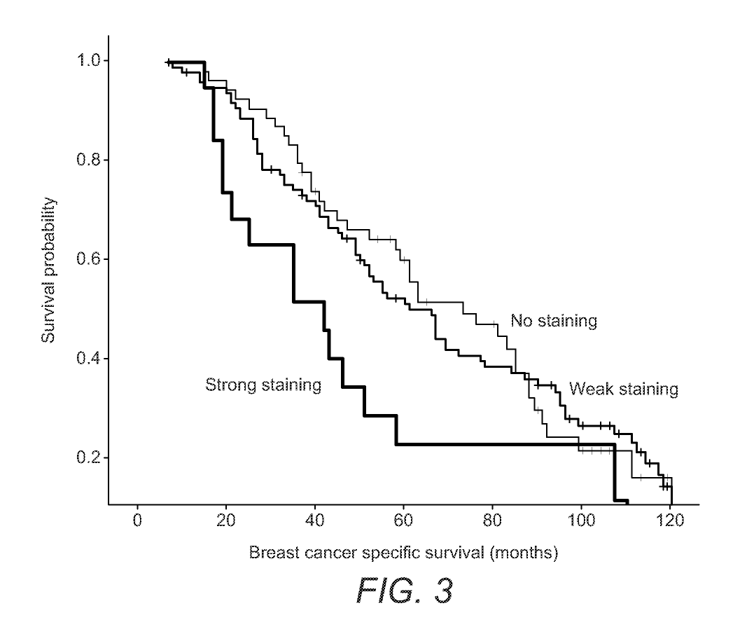

FIG. 3 shows Kaplan Meier plot showing a significant reduction in breast cancer specific survival in patients with high α1b adrenoceptor positive tumours (LR=7.628, p=0.022).

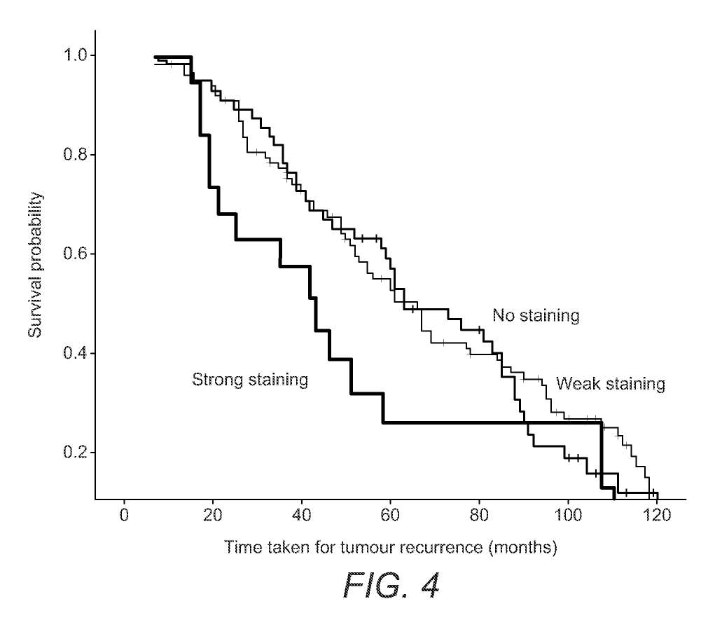

FIG. 4 shows Kaplan Meier plot showing a significant reduction in time for breast cancer recurrence in patients with strongly expressing α1b adrenoceptor positive tumours (LR=6.128, p=0.047).

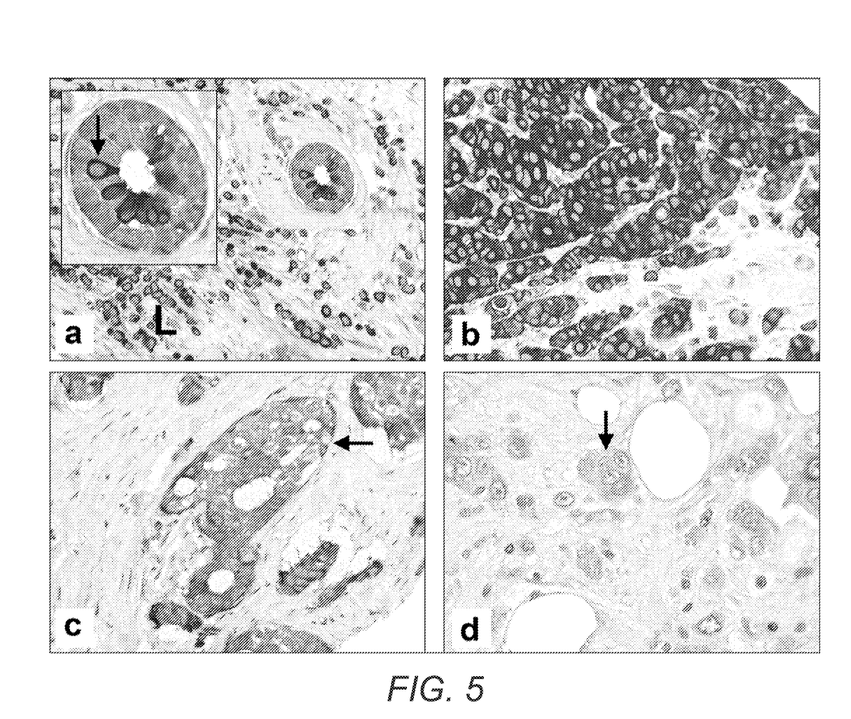

FIG. 5 shows immunostaining for β3₂ adrenoceptor protein showed it localised to the cytoplasm of (a) lobular breast cancer cells (L) and luminal cells (Inset: arrow) in normal glandular epithelium. Expression in ductal cancers varied from high (b) to moderate (c) and low (d) level expression.

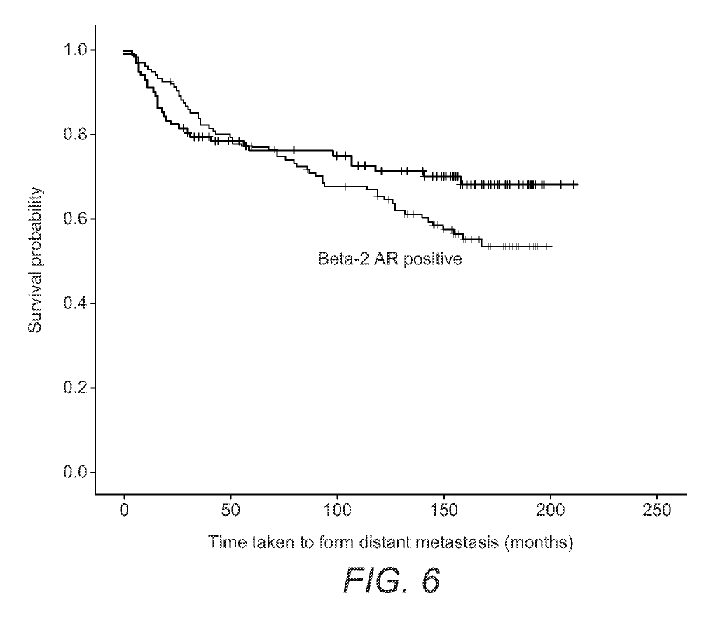

FIG. 6 shows Kaplan Meier plot showing a significant decline in survival for endocrine treated β2 adrenoceptor positive patients after approximately 60 months (LR=8.051, p=0.005); endocrine therapy is usually withdrawn after 60 months.

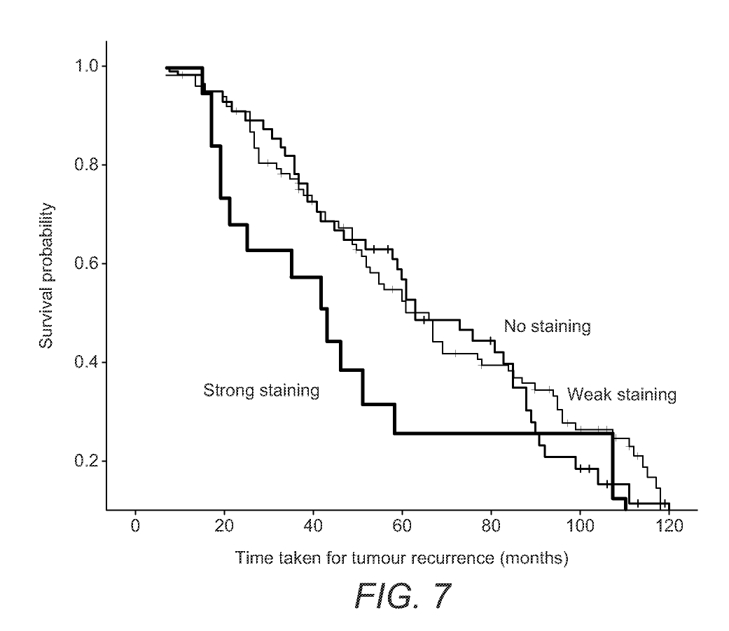

FIG. 7 shows diagram summarising the possible relationship between β2 adrenoceptor expression and oestrogen receptor stimulation in ER+ luminal-like breast cancer. The response to endocrine therapy and chemotherapy in alpha adrenoceptor positive is shown.

### ABBREVIATIONS

AR: androgen receptor/adrenoceptor
βAR: beta-adrenergic receptors
β₁AR: beta1-adrenergic receptors
β₂AR: beta2-adrenergic receptors
BCSS: breast cancer specific survival
CI: confidence interval
CK: cytokeratin
DFI: disease free interval
DM: distant metastasis
E: epinephrine
EGFR: epidermal growth factor receptor
ER: oestrogen receptor
NE: norepinephrine
PGR: progesterone receptor

### EXAMPLE 1

### Effect of Beta-Blocker Drug Therapy on Secondary Cancer Formation in Breast Cancer and Cancer Specific Survival

### Methods

### Patient Selection

Therapeutic drug and medical history was obtained for 466 patients with stage 1 and II primary operable breast carcinoma, aged 71 years or less, who presented consecutively to the Nottingham City Hospital between 1987 and 1994 as previously reported (El-Rehim et al Journal of Pathology 2004; 204:3 A-3A).

Patients were placed into one of three subgroups according to whether they received (1) beta-blocker treatment for hypertension, (2) other antihypertensive drug treatment, or were (3) normotensive. To qualify for subgroup (1) or (2) membership, patients needed to have been receiving antihypertensive therapy for at least 1 year prior to breast cancer diagnosis. This criterion was applied to minimise differences due to length of drug treatment; patients that received hypertensive drugs for less than 1 year were excluded because the primary objective tested was that beta-blockers may have a role in preventing metastasis formation in early stage breast cancer rather than eradicating or neutralising established primary and secondary cancers.

Patient's clinical and pathologic data was available including age, histologic tumour type, primary tumour size, lymph node status and histologic grade, Nottingham prognostic index (NPI), vascular invasion (VI), and radio/chemotherapy. Patients were considered for adjuvant therapy (AT) in a standardised scheduled on the basis of prognostic and predictive factor status including Nottingham Prognostic Index (NPI) (Blamey et al, European Journal of Cancer 2007; 43(10):1548-1555), oestrogen receptor-α (ERα) status, and menopausal status. Patients with a good prognostic index (NPI≦3.4) did not receive AT. Hormonal therapy (HT) was prescribed to patients with ERα+ tumours and NPI scores of >3.4 (moderate and poor prognostic groups). Pre-menopausal patients within the moderate and poor prognostic groups were candidates for CMF (Cyclophosphamide, Methotrexate, and 5-Flourouracil) chemotherapy; patients with ERα+ tumour were also offered HT. Conversely, postmenopausal patients with moderate or poor NPI and ERα+ were offered HT, while ERα− patients received CMF. Data has been accrued on a prospective basis for breast cancer specific survival (BCSS), disease free interval (DFI), formation of distant metastases (DM) and local tumour recurrence. BCSS was defined as the time (in months) from the date of the primary surgical treatment to the time of death from breast cancer. DFI was defined as the interval (in months) from the date of the primary surgical treatment to the first locoregional or distant metastasis. Mean follow-up time was 124 months for the study cohort.

### Univariate and Multivariate Statistics

The clinical outcome in three patient cohorts (beta-blocker drug treated, other antihypertensive drug treated, and non-hypertensive breast cancer groups) was tested using Kaplan-Meier plots with log rank test to assess significance including breast cancer specific survival, disease free interval, and distant metastasis formation. Other associations including tumour recurrence was tested using Chi square or Fishers exact test. Multivariate Cox regression analysis was used to evaluate the hazard ratio and any independent prognostic effect of the variables using 95% confidence interval (Version 15, SPSS Inc, IL, USA). A p-value of <0.05 was considered significant.

### Results

Clinical Correlations in Patients Treated with Beta-Blocker Drugs Compared to Other Hypertensive and Non-Hypertensive Breast Cancer Patients

Data was obtained for 466 breast cancer patients used in this study and their characteristics are shown in Tables 1a-d. A total of 92 (19.7%) patients had hypertension diagnosed prior to breast cancer diagnosis and were therapeutically treated using a range of antihypertensives including beta-blockers, ACE inhibitors, Ca2⁺ antagonists, imidazoline receptor antagonists or diuretics (Table 2). In particular, 43/92 (46.7%) of this hypertensive subgroup (median age 57 years, range 39-69) received beta-blocker drugs while the remaining 49/92 (53.3%) patients received other antihypertensive drugs. The latter group significantly differed in showing increased median age of 62 years (range 47-70) compared to the non-hypertensive patient subgroup whose median age was 54.5 years (range 28-71). In addition, the non-beta-blocker treated antihypertensive subgroup contained an increased proportion of post-menopausal patients (Table 1c) Otherwise, no significant difference was seen in tumour stage, tumour size, grade, type, vascular invasion, Nottingham Prognostic Index (NPI) or AT between the three patient subgroups.

Patients receiving beta-blockers showed a significant reduction in formation of distant metastasis (χ2=4.986, p=0.026) and tumour recurrence (χ2=13.091, p=0.001) compared to non-hypertensive BC control patients (Table 3).

Hypertensive Breast Cancer Patients Treated with Beta-Blockers Compared with Other Antihypertensive Drugs

Beta-blocker treated breast cancer patients differed significantly to patients receiving other antihypertensives (χ2=4.852, p=0.028) in showing reduced development of distant metastasis (5/43 (11.6%)) compared to their counterparts (15/49 (30.6%)) (Table 3). In addition, the beta-blocker treated subgroup showed significantly reduced tumour recurrence (χ2=7.264, p=0.026; Table 1b).

### Patient Outcome

Univariate analysis: Kaplan-Meier modelling with log rank testing showed beta-blocker treated patients had longer distant metastasis-free interval (Log rank (LR)=5.208, p=0.022) (FIG. 1a) and longer breast cancer specific survival (LR=6.479, p=0.011) (FIG. 1b), and longer disease free interval (LR=6.658, p=0.011) when compared to non-treated breast cancer patients.

Multivariate analysis: A multivariate Cox hazard model was used to determine the hazard ratio (HR) for predicting breast cancer specific survival and distant metastasis risk in patients receiving beta-blocker treatment compared to other significant breast cancer variables including tumour size, stage and grade. Patients receiving beta-blocker treatment had a 71% reduced risk of cancer-associated mortality (HR=0.291, CI=0.119-0.715, p=0.007). In addition, beta-blocker treated patients showed a 57% risk reduction in developing distant metastasis (HR=0.430, CI=0.200-0.926, p=0.031; Table 4) compared to non-treated breast cancer patients.

TABLE 1a
Characteristics for breast cancer patients therapeutically treated with
beta-blockers (BB) compared to control breast cancer (BC) patients,
excluding patients treated with other types of antihypertensive drugs.

| | | | | |
|---|---|---|---|---|
| | Number (%) | Number (%) | | |
| | Control BC | BB-Treated | | |
| Variable | patients | patients | χ2 | p-vlaue |

Patients' Age

| | | | | |
|---|---|---|---|---|
| <40 | 22 (5.9) | 1 (2.3) | 4.079 | 0.253 |
| 40-50 | 117 (31.3) | 9 (20.9) | | |
| 51-60 | 126 (33.7) | 20 (46.5) | | |
| >60 | 109 (29.1) | 13 (30.2) | | |

Primary tumour size

| | | | | |
|---|---|---|---|---|
| ≦1.5 cm | 134 (35.9) | 14 (33.3) | 0.111 | 0.740 |
| >1.5 cm | 239 (64.1) | 28 (66.7) | | |

Tumour stage

| | | | | |
|---|---|---|---|---|
| 1 | 250 (67.0) | 31 (73.8) | 1.176 | 0.555 |
| 2 | 89 (23.0) | 9 (21.4) | | |
| 3 | 34 (9.1) | 2 (4.8) | | |

Grade

| | | | | |
|---|---|---|---|---|
| 1 | 75 (20.2) | 7 (16.7) | 2.205 | 0.332 |
| 2 | 129 (34.7) | 11 (26.2) | | |
| 3 | 168 (45.2) | 24 (57.1) | | |

NPI

| | | | | |
|---|---|---|---|---|
| Poor | 43 (11.6) | 6 (14.3) | 0.270 | 0.874 |
| Moderate | 212 (57.1) | 23 (54.8) | | |
| Good | 116 (31.3) | 13 (31.0) | | |

Development of Recurrence

| | | | | |
|---|---|---|---|---|
| No | 220 (59.3) | 32 (74.4) | 13.091 | 0.001 |
| Positive | 151 (40.7) | 10 (23.3) | | |

Vascular invasion

| | | | | |
|---|---|---|---|---|
| Negative | 227 (61.5) | 27 (62.8) | 1.788 | 0.409 |
| Probable | 40 (10.8) | 2 (4.7) | | |
| Definite | 102 (27.6) | 14 (32.6) | | |

Tumour type

| | | | | |
|---|---|---|---|---|
| Ductal/NST | 200 (54.6) | 27 (62.8) | 7.433 | 0.283 |
| Lobular | 42 (11.5) | 4 (9.3) | | |
| Tubular and | 78 (21.3) | 8 (18.6) | | |
| Tubular mixed | | | | |
| Medullary | 11 (3.0) | 1 (2.3) | | |
| Other special types* | 10 (2.7) | 2 (4.7) | | |
| Mixed** | 24 (6.6) | 0 (0) | | |

Menopausal status

| | | | | |
|---|---|---|---|---|
| Premenopausal | 149 (39.8) | 9 (20.9) | 5.860 | 0.015 |
| Postmenopausal | 225 (60.2) | 34 (79.1) | | |

TABLE 1b
Characteristics for breast cancer patients therapeutically treated with
beta-blockers (BB) compared to breast cancer (BC) patients treated
with other antihypertensive drugs.

| | | | | |
|---|---|---|---|---|
| | Number (%) | | | |
| | BC treated with other | Number (%) | | |
| Variable | antihypertensives | BB-Treated | χ2 | p-vlaue |

Patients' Age

| | | | | |
|---|---|---|---|---|
| <40 | 0 (0) | 1 (2.3) | 6.507 | 0.089 |
| 40-50 | 10 (20.4) | 9 (20.9) | | |
| 51-60 | 13 (26.5) | 20 (46.5) | | |
| >60 | 26 (53.1) | 13 (30.2) | | |

Primary tumour size

| | | | | |
|---|---|---|---|---|
| ≦1.5 cm | 19 (39.6) | 14 (33.3) | 0.377 | 0.539 |
| >1.5 cm | 29 (60.4) | 28 (66.7) | | |

Tumour stage

| | | | | |
|---|---|---|---|---|
| 1 | 31 (66.0) | 31 (73.8) | 0.817 | 0.665 |
| 2 | 12 (25.5) | 9 (21.4) | | |
| 3 | 4 (8.5) | 2 (4.8) | | |

Grade

| | | | | |
|---|---|---|---|---|
| 1 | 12 (25.0) | 7 (16.7) | 2.215 | 0.330 |
| 2 | 16 (33.3) | 11 (26.2) | | |
| 3 | 20 (41.7) | 24 (57.1) | | |

NPI

| | | | | |
|---|---|---|---|---|
| Poor | 5 (10.2) | 6 (14.3) | 1.065 | 0.587 |
| Moderate | 24 (49.0) | 23 (54.8) | | |
| Good | 20 (40.8) | 13 (31.0) | | |

Development of Recurrence

| | | | | |
|---|---|---|---|---|
| No | 25 (51.0) | 32 (74.4) | 7.264 | 0.026 |
| Positive | 24 (49.0) | 10 (23.3) | | |

Vascular invasion

| | | | | |
|---|---|---|---|---|
| Negative | 30 (61.2) | 27 (62.8) | 1.811 | 0.404 |
| Probable | 6 (12.2) | 2 (4.7) | | |
| Definite | 13 (26.5) | 14 (32.6) | | |

Tumour type

| | | | | |
|---|---|---|---|---|
| Ductal/NST | 26 (53.1) | 27 (62.8) | 8.269 | 0.309 |
| Lobular | 3 (6.1) | 4 (9.3) | | |
| Tubular and | 15 (30.6) | 8 (18.6) | | |
| Tubular mixed | | | | |
| Medullary | 2 (4.1) | 1 (2.3) | | |
| Other special types* | 0 (0) | 2 (4.7) | | |
| Mixed** | 2 (4.1) | 0 (0) | | |

Menopausal status

| | | | | |
|---|---|---|---|---|
| Premenopausal | 12 (24.5) | 9 (20.9) | 0.165 | 0.685 |
| Postmenopausal | 37 (75.5) | 34 (79.1) | | |

TABLE 1c
Characteristics for breast cancer patients therapeutically treated with
antihypertensive drugs (excluding beta blockers) compared
to control breast cancer (BC) patients.

| | | | | |
|---|---|---|---|---|
| | | Number (%) | | |
| | Number (%) | BC treated | | |
| | Control BC | with other | | |
| Variable | patients | antihypertensives | χ2 | p-vlaue |

Patients' Age

| | | | | |
|---|---|---|---|---|
| <40 | 22 (5.9) | 0 (0) | 12.708 | 0.005 |
| 40-50 | 115 (30.7) | 10 (20.4) | | |
| 51-60 | 127 (34.0) | 131 (26.5) | | |
| >60 | 110 (29.4) | 26 (53.1) | | |

Primary tumour size

| | | | | |
|---|---|---|---|---|
| ≦1.5 cm | 134 (35.9) | 19 (39.6) | 0.246 | 0.620 |
| >1.5 cm | 239 (64.1) | 29 (60.4) | | |

Tumour stage

| | | | | |
|---|---|---|---|---|
| 1 | 249 (66.9) | 31 (66.0) | 0.069 | 0.966 |
| 2 | 89 (23.9) | 12 (25.5) | | |
| 3 | 34 (9.1) | 4 (8.5) | | |

Grade

| | | | | |
|---|---|---|---|---|
| 1 | 74 (19.9) | 12 (25.0) | 0.709 | 0.702 |
| 2 | 128 (34.4) | 16 (33.3) | | |
| 3 | 170 (45.7) | 20 (41.7) | | |

NPI

| | | | | |
|---|---|---|---|---|
| Poor | 43 (11.6) | 5 (10.2) | 1.917 | 0.384 |
| Moderate | 213 (57.4) | 24 (49.0) | | |
| Good | 115 (31.0) | 20 (40.8) | | |

Development of Recurrence

| | | | | |
|---|---|---|---|---|
| No | 218 (58.8) | 25 (51.0) | 1.063 | 0.302 |
| Positive | 153 (41.2) | 24 (49.0) | | |

Vascular invasion

| | | | | |
|---|---|---|---|---|
| Negative | 228 (61.8) | 30 (61.2) | 0.057 | 0.972 |
| Probable | 41 (11.1) | 6 (12.2) | | |
| Definite | 100 (27.1) | 13 (26.5) | | |

Tumour type

| | | | | |
|---|---|---|---|---|
| Ductal/NST | 201 (54.9) | 26 (53.1) | 12.228 | 0.093 |
| Lobular | 42 (11.5) | 3 (6.1) | | |
| Tubular and | 78 (21.3) | 15 (30.6) | | |
| Tubular mixed | | | | |
| Medullary | 11 (3.0) | 2 (4.1) | | |
| Other special types* | 9 (2.5) | 0 (0) | | |
| Mixed** | 24 (6.6) | 2 (4.1) | | |

Menopausal status

| | | | | |
|---|---|---|---|---|
| Premenopausal | 147 (39.9) | 12 (24.5) | 4.053 | 0.044 |
| Postmenopausal | 229 (60.7) | 37 (75.5) | | |

TABLE 1d
Characteristics for breast cancer patients therapeutically treated with
beta-blockers (BB) compared to all other patients including
those receiving other antihypertensive drug treatment.

| | | | | |
|---|---|---|---|---|
| | Number (%) | | | |
| | All other | Number (%) | | |
| Variable | patients | BB-Treated | χ2 | p-vlaue |

Patients' Age

| | | | | |
|---|---|---|---|---|
| <40 | 22 (5.2) | 1 (2.3) | 3.926 | 0.270 |
| 40-50 | 127 (30.1) | 9 (20.9) | | |
| 51-60 | 139 (32.9) | 20 (46.5) | | |
| >60 | 135 (31.9) | 13 (30.2) | | |

Primary tumour size

| | | | | |
|---|---|---|---|---|
| ≦1.5 cm | 153 (36.3) | 14 (33.3) | 0.150 | 0.699 |
| >1.5 cm | 268 (63.7) | 28 (66.7) | | |

Tumour stage

| | | | | |
|---|---|---|---|---|
| 1 | 281 (66.9) | 31 (73.8) | 1.190 | 0.552 |
| 2 | 101 (24.0) | 9 (21.4) | | |
| 3 | 38 (9.0) | 2 (4.8) | | |

Grade

| | | | | |
|---|---|---|---|---|
| 1 | 87 (20.7) | 7 (16.7) | 2.368 | 0.306 |
| 2 | 145 (34.5) | 11 (26.2) | | |
| 3 | 188 (44.8) | 24 (57.1) | | |

NPI

| | | | | |
|---|---|---|---|---|
| Poor | 48 (11.4) | 6 (14.3) | 0.305 | 0.859 |
| Moderate | 236 (56.2) | 23 (54.8) | | |
| Good | 136 (32.4) | 13 (31.0) | | |

Development of Recurrence

| | | | | |
|---|---|---|---|---|
| No | 244 (58.2) | 33 (78.6) | 6.854 | 0.010 |
| Positive | 175 (41.8) | 9 (21.4) | | |

Vascular invasion

| | | | | |
|---|---|---|---|---|
| Negative | 257 (61.5) | 27 (62.8) | 1.877 | 0.391 |
| Probable | 46 (11.0) | 2 (4.7) | | |
| Definite | 115 (27.5) | 14 (32.6) | | |

Tumour type

| | | | | |
|---|---|---|---|---|
| Ductal/NST | 233 (55.5) | 28 (65.1) | 3.534 | 0.832 |
| Lobular | 46 (10.9) | 4 (9.3) | | |
| Tubular and | 92 (21.9) | 7 (16.3) | | |
| Tubular mixed | | | | |
| Medullary | 13 (3.1) | 1 (2.3) | | |
| Other special types* | 10 (2.4) | 2 (4.6) | | |
| Mixed** | 26 (6.2) | 1 (2.3) | | |

Menopausal status

| | | | | |
|---|---|---|---|---|
| Premenopausal | 160 (37.9) | 11 (25.6) | 2.553 | 0.110 |
| Postmenopausal | 262 (62.1) | 32 (74.4) | | |

*Includes Mucoid, invasive cribriform and invasive papillary carcinoma.
**Includes ductal/NST mixed with lobular or special types.

TABLE 2
Beta-blocker and other therapeutic drugs were used to treat breast
cancer patients for pre-existing hypertension.

| Drug | Patient Numbers |
|---|---|
| Beta-blockers | |
| Atenolol | 25 |
| Propranolol | 7 |
| Bisoprolol | 7 |
| Timolol | 4 |
| Subtotal | 43 |
| Other drug treatments | |
| Captopril (ACE − Inhibitor) | 6 |
| Ramipril (ACE − Inhibitor) | 4 |
| Enalapril (ACE − Inhibitor) | 3 |
| Lisinopril (ACE − Inhibitor) | 3 |
| Nifedipine (Ca2 + antagonist) | 5 |
| Amlodipine (Ca2 + antagonist) | 5 |
| Nicardipine (Ca2 + antagonist) | 1 |
| Moxonidine (Imidazoline receptor antagonist) | 1 |
| Bendrofluazide (Diuretic) | 21 |
| Subtotal | 49 |
| Total | 92 |

TABLE 3
Two hypertensive breast cancer (BC) patient groups comprising those
treated with beta-blockers and non-beta blocker antihypertensive
drugs were compared with a non-hypertensive non-treated BC group
for formation of distant metastases.

| Metastasis | Treatment in breast | | | |
|---|---|---|---|---|
| formation | cancer groups | | χ² | P-value |
| | Non- | Beta-blocker | | |
| | hypertensive | treated | | |
| | patients (%) | hypertensive (%) | | |
| Negative | 271 (72.7) | 38 (88.4) | 4.986 | 0.026 |
| Positive | 102 (27.3) | 5 (11.6) | | |
| | Other treated | Beta-blocker | | |
| | anti- | treated | | |
| | hypertensive | patients | | |
| | patients (%) | (%) | | |
| Negative | 34 (69.4) | 38 (88.4) | 4.852 | 0.028 |
| Positive | 15 (30.6) | 5 (11.6) | | |
| | Non | Other anti- | | |
| | hypertensive | hypertensive | | |
| | BC patients (%) | patients | | |
| Negative | 271 (72.7) | 34 (69.4) | 0.231 | 0.631 |
| Positive | 102 (27.3) | 15 (30.6) | | |

TABLE 4
The effect of beta-blocker treatment on breast cancer specific
survival (BCSS) and distant metastasis (DM) formation was
compared with tumour size, grade and stage to determine the
relative risk (Hazard Ratios (HR)) in BC patients.

| | | | 95% Confidence Interval | |
|---|---|---|---|---|
| Parameter | HR | p-value | Lower | Upper |
| BCSS | | | | |
| Tumour size | 1.985 | 0.004 | 1.248 | 3.159 |
| Tumour grade | 1.904 | <0.001 | 1.435 | 2.526 |
| Tumour stage | 1.565 | <0.001 | 1.218 | 2.011 |
| beta-blocker treatment | 0.291 | 0.007 | 0.119 | 0.715 |
| DM | | | | |
| Tumour size | 1.916 | 0.005 | 1.221 | 3.005 |
| Tumour grade | 1.519 | 0.002 | 1.171 | 1.971 |
| Tumour stage | 1.624 | <0.001 | 1.270 | 2.076 |
| beta-blocker treatment | 0.430 | 0.031 | 0.200 | 0.926 |

This study showed beta-blocker therapy significantly reduces distant metastases, cancer recurrence, and cancer-specific mortality in breast cancer patients suggesting a novel role for beta-blocker therapy.

Tumour metastasis is a complex process and is associated with generally poor clinical outcome. Therapeutic strategies are needed that can prevent its occurrence, thereby prolonging patient life. The present study was performed to validate the putative role of beta-blocker adrenergic receptor antagonists in retarding the progress of breast cancer disease by reducing metastasis formation. We performed an epidemiological study of breast cancer patients with long term clinical follow up (>10 years) and showed that patients receiving antihypertensive beta-blocker drugs significantly benefit by a 57% reduction in distant metastasis formation and a 71% reduced risk of dying from breast cancer compared to control patients.

It is increasingly being recognised that stress can promote cancer progression through an indirect effect on the immune system. Moreover, a biological mechanism of action has been proposed for the involvement of catecholamine stress hormones. It has been shown that norepinephrine can directly stimulate tumour cell migration and this effect is mediated by the beta-adrenergic receptor, β2AR. High levels of β₂AR have been reported in human cell lines and tumour samples and importantly, we have shown that cell migration in a number of cancer models is inhibited by the beta-blocker adrenergic receptor antagonist, propranolol. The aim of the current study was to translate these findings into a clinical setting by testing the hypothesis that breast cancer patients receiving beta-blockers for pre-existing hypertension would show a significant reduction in tumour metastasis with a consequent reduction in mortality.

In line with expectations, antihypertensive drug treatment was more frequently prescribed in older post-menopausal patients with no significant difference seen between the beta-blocker and other antihypertensive treatment subgroups. The beta-blocker treated subgroup was shown to have significantly (57%) reduced risk of developing distant metastasis and tumour recurrence compared to patients receiving either no, or other types of, antihypertensive drugs. As a consequence of this, beta-blocker treated patients showed a significant increase in breast cancer specific survival and increased disease free interval. To the best of our knowledge, this is the first report highlighting the in vivo therapeutic benefits of beta-blockers in breast cancer patients, supporting the biological mechanism of norepinephrine-induced cell migration mediated by adrenergic receptor activation as shown in cell lines and animal models. These findings support recent epidemiological cancer studies that have shown adrenoceptor antagonists have the potential for providing a novel clinically effective therapeutic strategy in treating several different cancer types.

Several limitations apply to our epidemiological study including patient population size and factors that were not controlled for in using an existing patient dataset. In mitigation, the beta-blocker and other hypertensive-treated cohort were of approximately the same size and together accounted for approximately 20% of the total patient cohort. In addition, all patient subgroups were evenly matched for adjuvant therapy and age making it unlikely that this was the reason for the significant benefits seen in the beta-blocker group. A further possible limitation is that it is unknown (and impossible to establish) how long patients had breast cancer prior to a formal diagnosis, and so the duration required for beta-blockers to prevent development of metastases can not be calculated from this type of study. Some of these limitations can be addressed by performing a much larger epidemiological study, leading onto a randomised controlled clinical trial.

The current study suggests that adrenoceptor antagonists have the potential for retarding breast cancer progression and improving clinical outcome. Additional studies are needed to assess the protein expression of adrenoceptors in breast and other cancer types to test if they can be used as prognostic and predictive biomarkers in determining clinical outcome and likely response to antagonist treatment.

### EXAMPLE 2

### Study on Alpha and Beta Adrenergic Receptor (Ar) Protein Expression and Association with Poor Clinical Outcome in Breast Cancer: an Immunohistochemical Study

We investigated adrenoceptor protein expression in clinical breast tumours and its association with disease progression and prognosis. In the present study, immunohistochemistry was used to assess protein levels of two alpha (α1b, α2c) and one beta (β₂) adrenoceptors in malignant breast tissue microarrays. Associations between adrenoceptor expression and clinical outcome, and other tumour-relevant biological markers, were statistically assessed.

### Methods

Immunohistochemistry on tissue microarrays was used to characterise α1b, α2c and β₂2 adrenoceptor protein expression in operable breast tumours. Associations with tumour-relevant biological markers and clinical outcome were statistically assessed.

### Materials and Methods

The protein expression of two alpha (α1b, α2c) and one beta-isoform (β₂) adrenoceptors was characterised in formalin fixed paraffin embedded (FFPE) tissue microarrays of breast cancer tissue to establish the relationship with other cancer-relevant biological markers and their association with clinicopathologic features. The cut-offs used for categorising the various biomarkers have been previously described (Abdel-Fatah et al EJC Supplements 2009, 7(2):264-264; El-Rehim et al. Int J Cancer 2005, 116(3):340-350; El-Rehim et al. J Pathol 2004, 204:3 A-3A.; Habashy et al. Eur J Cancer 2008, 44(11):1541-1551). HER2 scoring was performed using the Hercept tests guidelines (DakoCytomation, Cambridge, UK)

### Patient Selection

Tissue microarray (TMA) slides from the Nottingham Breast Cancer group were used in this study comprising approximately 750 patients from women aged 70 or less derived from the Nottingham Tenovus Primary Breast Carcinoma Series (1986 and 1999). This well characterized resource contains information on patients' clinical and pathological data including histologic tumour type, primary tumour size, lymph node status, histologic grade, and data on other breast cancer relevant biomarkers. Patients within the good prognostic group (Nottingham Prognostic Index (NPI)≦3.4) did not receive adjuvant therapy (AT). Hormonal therapy (HT) was prescribed to patients with ER-α+ tumours and NPI scores of >3.4 (moderate and poor prognostic groups). Pre-menopausal patients within the moderate and poor prognostic groups were candidates for CMF (Cyclophosphamide, Methotrexate, and 5-Flourouracil) chemotherapy. Conversely, postmenopausal patients with moderate or poor NPI and ER-α+ were offered HT, while ER-α− patients received CMF if fit. Survival data including survival time, disease-free interval (DFI) and development of loco-regional and distant metastases (DM) were maintained on a prospective basis. Median follow up was 124 months (range 1 to 233). Breast cancer specific survival (BCSS) was defined as the time (in months) from the date of the primary surgical treatment to the time of death from breast cancer. DFI was defined as the interval (in months) from the date of the primary surgical treatment to the first loco-regional or distant metastasis. This study was approved by the Nottingham Research Ethics Committee 2 under the title “Development of a molecular genetics classification of breast cancer”. Patients consented to tissue samples being used for research purposes.

### Immunohistochemistry for the Detection of Alpha and Beta Adrenoceptor Protein

Immunohistochemistry (IHC) was performed using a DakoCytomation Techmate 500 plus (DakoCytomation, Cambridge, UK) instrument with a linked streptavidin biotin technique and DAB chromogen as previously described. Primary antibodies were optimised on full face FFPE sections and TMAs of breast cancer with appropriate positive control kidney and heart tissue. Negative controls comprised omission of the primary antibody and substitution with an inappropriate primary antibody of the same Ig class. After dewaxing, sections were subjected to microwave antigen retrieval using either 0.01M EDTA, pH9 (α1b) or 0.01M citrate buffer, pH6 (α2c, β₂) and then immunostained using the optimised dilution of adrenoceptor antibody. The rabbit polyclonal α1bAR (ab13297, Abcam, Cambs, UK) and α2cAR (ab46536, Abcam) antibodies were used at a dilution of 1:50 and 1:750 respectively. The rabbit polyclonal β₂AR (ab13163, Abcam) antibody was used at a dilution of 1:450.

Staining intensity was subjectively assessed (DGP & HOH) for each marker according to a three point scoring system comprising 0 (negative), 1 (weak) or 2 (strong) expression confined to only malignant epithelial tissue component. Cases were scored without knowledge of patient outcome.

### Univariate and Multivariate Statistics

The results have been reported according to REMARK criteria that establish a framework for reporting tumour marker prognostic studies (McShane et al. Breast Cancer Res Treat 2006, 100(2):229-235). The association between adrenoceptor expression and other tumour-relevant markers was assessed using Chi square or Fishers exact test. Association with clinical outcome including BCSS, DFI, DM formation, and local tumour recurrence was modelled using Kaplan-Meier plots (Version 15, SPSS Inc, IL, USA) with the log rank (Mantel-Cox) test. A p-value of less than 0.05 was deemed significant with 95% confidence intervals.

### Results

Strong α1b expression occurred in large high grade (p<0.0001), HER2+ (p<0.0001) or basal-like (CK5/6, p=0.0005; CK14, p=0.0001; EGFR, p=0.003) cancers, showing increased proliferation (Mib1, p=0.002), decreased apoptosis (Bc12, p<0.0001) and poor NPI membership (p=0.001). α1b expression correlated with poor cancer specific survival (LR=7.628, p=0.022) and tumour recurrence (LR=6.128, p=0.047). Strong α2c was over-expressed in high grade (p=0.007), HER3+ (p=0.002) and HER4+ (p<0.0001) cancers with borderline increase in EGFR, p53 and MIB1 proteins, and inverse association with hormonal (PgR, p=0.002) phenotype. In contrast, strong β₂ expression occurred in small-size, luminal-like (ER+, p<0.001) tumours of low grade (p<0.001) and lymph node stage (p=0.027) that showed poor prognosis when hormonal treatment was withheld. Adrenoceptors were not found to be independent predictors of clinical outcome.

Alpha1b (α1b AR) adrenoceptor protein expression Informative data was obtained on 544 patients with a median age of 56 years. Strong cytoplasmic α1bAR expression was significantly more prevalent in larger tumours of high histologic grade, and poor Nottingham Prognostic Index (NPI) (FIG. 2). Patients with strong α1bAR expression had significantly increased frequency of ductal type breast cancers with an absence of lobular cancers compared to patients lacking α1bAR expression (Table 5). α1bAR expression showed no significant association with clinical stage, menopausal status or patients' age.

Highly significant inverse associations were found for strong α1bAR expression and luminal cell-associated markers including CK18 (χ2=7.228, p=0.027), ER (χ2=20.345, p<0.0001), PgR (χ2=18.098, p=0.0001), CD71 (χ2=15.658, p=0.0004) and FOXA1 (χ2=8.032, p=0.018) (Table 6). Conversely, strong α1bAR expressing patients showed a significant positive association in having HER2+ tumours (χ2=24.468, p<0.001). In addition, strong α1bAR expression positively correlated with biological markers of aggressive phenotype including the cell adhesion molecule, P-cadherin, and basal-like tumour markers CK5/6 (χ2=15.219, p=0.0005), CK14 (χ2=22.585, p=0.0001) and EGFR (χ2=11.452, p=0.003). Furthermore, the same patient group showed significant association with increased expression of the proliferation marker, MIB1 (χ2=12.987, p=0.002) and reduced expression of the anti-apoptotic marker Bc12 (χ2=22.107, p<0.0001) (Table 6). No significant association was seen for α1bAR expression and E-cadherin, HER3, HER4, and BRCA1 protein expression.

Clinical outcome was studied over a 10 year period using Kaplan-Meier models and showed significant associations between strong α1bAR expression and BCSS (LR=7.628, p=0.022) (FIG. 3) and tumour recurrence (LR=6.128, p=0.047) (FIG. 4), but not with DM formation (LR=4.136, p=0.126) and DFI (LR=4.476, p=0.107). Multivariate analysis using Cox regression showed α1bAR expression (HR=1.154, p=0.371, CI (95%)=0.843-1.579) is not an independent predictor of survival compared to tumour size, stage and grade (data not shown).

Alpha2c (α2c AR) adrenoceptor protein expression

Informative data was obtained on 541 patients with a median age of 55 years. Strong cytoplasmic α2cAR expression was significantly more prevalent in tumours of high histologic grade and showed a positive association with post-menopausal status (FIG. 2). α2cAR positive tumours were less abundant among the lobular and tubular type cancers, but conversely, were more frequent in medullary type cancers. α2cAR expression showed no significant association with tumour size, clinical stage, Nottingham Prognostic Index (NPI), or vascular invasion (Table 7).

Highly significant inverse associations were found between strong α2cAR expression and the hormonal marker, PgR (χ2=12.399, p=0.002) but a positive association with the ER-related marker, CD71 (χ2=10.817, p=0.004). Positive associations were seen between strong α2cAR positivity and HER3 (χ2=12.674, p=0.002) and HER4 (χ2=30.082, p<0.001). p53 (χ2=5.301, p=0.071) was over-expressed in α2cAR positive breast cancers and the proliferation marker MIB1 (χ2=5.888, p=0.053) showed borderline significance (Table 8). No significant associations were found between α2cAR protein expression and CK18, ER, FOXA1, HER2, BRCA1, P-cadherin, E-cadherin, EGFR, CK5/6, CK14, P53, and Bc12 (Table 8).

Clinical outcome was studied over a 10 year period using Kaplan-Meier models and showed no significant associations between strong α2cAR expression and BCSS (LR=0.962, p=0.618), tumour recurrence (LR=1.817, p=0.403), DM formation (LR=1.348, p=0.510) and DFI (LR=804, p=0.669) (data not shown).

### Beta-2 (β₂AR) Adrenoceptor Protein Expression

Informative data for β₂AR was obtained on 690 patients. Strong β₂AR expression was significantly more prevalent in smaller tumours of low histologic grade, clinical stage, and good Nottingham Prognostic Index (NPI) and showed a positive association with increasing age. In addition, patients with strong β₂AR expression had a significantly increased frequency (31%) of tubular breast cancers compared to patients lacking β2AR expression (6.3%) (Table 9). No significant association was found between β2AR expression and vascular invasion or menopausal status.

In normal breast tissue components, strong cytoplasmic β₂AR staining was seen localised to the luminal cell layer (FIG. 5). In tumours, highly significant associations were found between strong β₂AR expression and luminal cell-associated markers including CK18 (χ2=86.243, p<0.0001), ER (χ2=52.588, p<0.001), PgR (χ2=44.502, p<0.001), AR (χ2=37.932, p<0.001) and FOXA1 (χ2=6.309, p=0.043) (Table 10). Altogether, 305/383 (79.6%) patients had tumours co-expressing oestrogen receptor and β₂AR. Conversely, strong β₂AR expression was negatively correlated with biological markers of aggressive phenotype including the cell adhesion molecule P-cadherin, HER3 and -4, and the basal-like tumour marker CK5/6. The same patient group stained positive for wild-type BRCA1 suggesting an absence of mutation in this oncogene. In addition, strongly positive β₂AR patients were less likely to have p53 mutation, indicated by reduced levels of (mutated) p53 protein, and showed less abundant proliferation features as evidenced by reduced levels of Mib1 (Table 10).

Clinical outcome was determined using Kaplan-Meier models. No significant association was found between strong β₂AR expression and BCSS (LR=0.424, p=0.809), DM formation (LR=0.968, p=0.616) and tumour recurrence (LR=1.785, p=0.410). The Kaplan-Meier plot showed patients with strong β₂AR had improved, albeit insignificant, survival compared to patients with low level expression.

### Outcome According to Systemic Therapy Groups

Patients with high levels of PAR showed no significant association with BCSS (LR=0.036, p=0.851), DM (LR=2.108, p=0.147) or tumour recurrence (LR=0.013, p=0.910) when treated with tamoxifen (n=138) over a 10 year follow up period. However, the Kaplan-Meier plot shows a reduction in time taken to form metastases in PAR positive patients treated with tamoxifen, giving poor prognosis, after about 60 months (FIG. 6). In only considering patients surviving after 60 months, PAR positive patients show a significant reduction in the time taken to form metastases (LR=8.051, p=0.005). Non-significant associations were seen between PAR and BCSS (LR=0.468, p=0.494), DM (LR=0.975, p=0.323) or tumour recurrence (LR=1.712, p=0.191) when considering the effect of chemotherapy treatment (n=91).

Kaplan-Meier plots of patients with high levels of α1bAR and treated with endocrine therapy (n=182) showed worse prognosis than the whole patient series but the finding was non-significant for BCSS (LR=1.963, p=0.375), DM (LR=0.259, p=0.879) and tumour recurrence (LR=2.088, p=0.352). Patients treated with chemotherapy (n=91) showed similar prognosis to the whole patient series in showing no significant association between high α1bAR expression and BCSS (LR=0.140, p=0.932), DM (LR=0.048, p=0.976) or tumour recurrence (LR=2.170, p=0.338).

Kaplan-Meier plots of endocrine-treated patients (n=187) with high levels of α2cAR showed worse prognosis, reaching significance for DM formation (LR=7.189, p=0.027) and borderline significance for BCSS (LR=5.521, p=0.063), but not for tumour recurrence (LR=1.348, p=0.510). Patients expressing high levels of α2cAR showed improved (but insignificant) DM (LR=1.523, p=0.467) when treated with chemotherapy (n=122) compared to the whole patient series, but otherwise similar BCSS (LR=1.717, p=0.424) and tumour recurrence (LR=1.019, p=0.601) to that seen in the whole patient series.

### Conclusions

Alpha1b and α2c AR is over-expressed in basal-like breast tumours of poor prognosis.

Strong β₂ adrenoceptor expression is seen in patients with a luminal (ER+) tumour phenotype and good prognosis, due to benefits derived from hormonal therapy. These findings suggest a role for targeted therapy using adrenoceptor antagonists.

In the present study we showed that the protein expression of three types of adrenoceptor can be used to predict clinical outcome and possible candidacy for adrenoceptor antagonist therapy.

Two alpha (α1b, α2c) and one beta β₂) adrenoceptor isoforms were selected for investigation because of their reported cancer-promoting functions.

In contrast, β2AR is reported to be highly expressed in breast cancer cell lines (Masur et al. Cancer Res 2001, 61(7):2866-2869; Slotkin et al. Breast Cancer Res Treat 2000, 60(2):153-166; Vandewalle et al J Cancer Res Clin Oncol 1990, 116(3):303-306) and tumour samples (Shang et al. Journal of Oral Pathology & Medicine 2009, 38(4):371-376), and is a key target for beta-adrenoceptor antagonists (beta-blockers). This was confirmed in the current study where it was found in small luminal-like (ER+) cancers of low clinical stage, low proliferation and improved prognosis, supporting the proposal that β2AR expression is regulated by the hormones oestrogen and progesterone. Ovariectomized rats show decreased β2AR expression, demonstrating its dependency on endocrine hormones (Marchetti et al. Endocrinology 1990, 126(1):565-574) but in another study, a weak correlation between β₂AR expression with hormonal receptors was reported for human breast cancer (Draoui et al. Anticancer Res 1991, 11(2): 677-680).

Our data offer a possible explanation for the cancer-enhancing role of β2AR in promoting cell migration and proliferation (Drell et al. Breast Cancer Res Treat 2003, 80(1):63-70; Schuller et al: Neuronal Activity in Tumor Tissue. vol. 39; 2007: 45-6) and its contradictory association with favourable patient prognosis reported here. The latter could be due to the effectiveness of the estrogen antagonist, tamoxifen. Close inspection of the Kaplan-Meier plot for breast cancer-specific survival in β2AR positive patients treated with tamoxifen demonstrates good prognosis during the first 5 years of treatment (FIG. 4) but at about 60 months, this trend is reversed. Subsequently, patients develop significantly poor prognosis and this coincides with tamoxifen withdrawal after about 5 years' treatment. Further studies are needed to better understand the relationship between β2 adrenoceptor and its regulation by estrogen, and whether β2 antagonists could be used as an adjuvant therapy especially in patients that are resistant to tamoxifen (FIG. 5).

Alpha adrenoceptor proteins were over-expressed in tumours with a more aggressive biological phenotype characteristic of their membership to the basal-like molecular classification group. As such, α1bAR tumours showed increased cell proliferation and poor clinical outcome but no significant association with development of metastases, Patients with α1bAR positive tumours were found to be non-responsive to hormonal (tamoxifen) treatment.

Patients with high levels of α2c adrenoceptor shared similar characteristics to α1bAR positive patients in being more abundant in non-luminal (ER/PGR negative) high grade breast cancers characterised by high cell proliferation, mutated p53, and poor prognosis. α1b adrenoceptor positive patients showed no significant benefit with tamoxifen therapy either but showed a trend toward beneficial response to chemotherapy.

The current study has shown adrenoceptor proteins are differentially expressed in breast cancer and in some instances can predict response to hormonal and chemotherapeutic treatment.

### Conclusion

This study has shown that alpha and beta-adrenoceptors are differentially over-expressed in different types of breast tumour and are associated with poor prognosis. In particular, high α1b and α2c adrenoceptor expression appear in high grade basal-like cancers, with α1b positive tumours showing a significant association with reduced cancer-specific survival and increased tumour recurrence. High β₂ adrenoceptor expression was found in ER hormonally positive patients with good 5 year post-diagnosis prognosis. However, the prognosis was reversed after 5 years and might be explained by removal of tamoxifen-induced therapeutic benefits.

TABLE 5
Characteristics for patients investigated for α1b
adrenoceptor protein expression.

| | Number (%) | Number (%) | Number (%) | | |
|---|---|---|---|---|---|
| | Alpha1b ADR | Alpha1b ADR | Alph1b ADR | | |
| | negative | weak positive | strong positive | | |
| Variable | expression | expression | expression | χ² | P-value |
| Patients' Age | | | | | |
| <40 | 14 (6.5) | 21 (7.8) | 6 (10) | 4.758 | 0.575 |
| 40-50 | 72 (33.6) | 73 (27) | 17 (28.3) | | |
| 51-60 | 70 (32.7) | 88 (32.6) | 16 (26.7) | | |
| >60 | 58 (27.1) | 88 (32.6) | 21 (35) | | |
| Primary tumour size | | | | | |
| ≦1.5 cm | 98 (46.2) | 83 (30.9) | 15 (25) | 15.808 | 0.0004 |
| >1.5 cm | 114 (53.8) | 186 (69.1) | 75 (75) | | |
| Lymph node Stage | | | | | |
| 1 | 159 (70) | 164 (60.7) | 35 (58.3) | 6.310 | 0.177 |
| 2 | 48 (22.5) | 72 (26.7) | 17 (28.3) | | |
| 3 | 16 (7.5) | 34 (12.6) | 8 (13.3) | | |
| Grade | | | | | |
| 1 | 52 (24.5) | 48 (17.8) | 8 (13.3) | 31.125 | 2.886e−6 |
| 2 | 83 (31.2) | 83 (30.9) | 7 (11.7) | | |
| 3 | 77 (36.3) | 138 (51.3) | 45 (75) | | |
| NPI | | | | | |
| Poor | 23 (10.8) | 47 (17.5) | 12 (20) | 19.203 | 0.001 |
| Moderate | 101 (47.6) | 148 (55) | 38 (63.3) | | |
| Good | 88 (41.5) | 74 (27.5) | 10 (16.7) | | |
| Development of DM | | | | | |
| No | 147 (69) | 181 (67.5) | 41 (68.3) | 0.120 | 0.942 |
| Positive | 66 (31) | 87 (32.5) | 19 (31.7) | | |
| Development of | | | | | |
| Recurrence | | | | | |
| No | 120 (57.1) | 143 (54) | 35 (58.3) | 0.670 | 0.715 |
| Positive | 90 (42.9) | 122 (46) | 25 (41.7) | | |
| Vascular invasion | | | | | |
| No | 132 (62.3) | 154 (57.2) | 30 (51.7) | 7.308 | 0.120 |
| Probable | 25 (11.8) | 21 (7.8) | 5 (8.6) | | |
| Definite | 55 (25.9) | 94 (34.9) | 23 (39.7) | | |
| Tumour type | | | | | |
| Ductal/NST | 90 (42.3) | 173 (64.8) | 45 (76.3) | 68.730 | 5.532e−10 |
| Lobular | 40 (18.8) | 6 (2.2) | 0 (0) | | |
| Tubular and | 55 (25.8) | 64 (24) | 6 (10.2) | | |
| Tubular mixed | | | | | |
| Medullary | 8 (3.8) | 4 (1.5) | 3 (5.1) | | |
| Other special types* | 2 (0.9) | 4 (1.5) | 1 (1.7) | | |
| Mixed** | 16 (7.5) | 14 (5.2) | 4 (6.8) | | |
| Menopausal status | | | | | |
| Premenopausal | 81 (37.9) | 104 (38.5) | 23 (38.3) | 0.023 | 0.989 |
| Postmenopausal | 133 (62.1) | 166 (61.5) | 37 (61.7) | | |

*Includes Mucoid, invasive cribriform and invasive papillary carcinoma.
**Includes ductal/NST mixed with lobular or special types.

TABLE 6
Associations between α1b adrenoceptor protein expression
and tumour relevant markers.

| | Number | Number | Number | | |
|---|---|---|---|---|---|
| | (%) Alpha1B | (%) | (%) | | |
| | ADR | Alpha1B | Alpha1B | | |
| | negative | ADR weak | ADR strong | | |
| Variable | expression | expression | expression | χ² | P-value |
| ER | | | | | |
| Negative | 58 (29.6) | 83 (33.2) | 35 (61.4) | 20.345 | 3.8197e−5 |
| Positive | 138 (70.4) | 167 (66.8) | 22 (38.6) | | |
| PgR | | | | | |
| Negative | 78 (39.4) | 113 (45.4) | 40 (71.4) | 18.098 | 0.0001 |
| Positive | 120 (60.6) | 136 (54.6) | 16 (28.6) | | |
| AR | | | | | |
| Negative | 66 (35.6) | 93 (39.6) | 28 (51.9) | 4.461 | 0.107 |
| Positive | 118 (64.1) | 142 (60.4) | 26 (48.1) | | |
| CD71 | | | | | |
| Negative | 60 (44.1) | 56 (31.6) | 3 (9.1) | 15.658 | 0.0004 |
| Positive | 76 (55.9) | 121 (68.4) | 30 (90.9) | | |
| FOXA1 | | | | | |
| Negative | 52 (39.7) | 100 (55.6) | 17 (54.8) | 8.032 | 0.018 |
| Positive | 79 (60.3) | 80 (44.4) | 14 (45.2) | | |
| CK18 | | | | | |
| Negative | 29 (16.9) | 30 (12.9) | 15 (27.8) | 7.228 | 0.027 |
| Positive | 143 (83.1) | 202 (87.1) | 39 (72.2) | | |
| HER2 | | | | | |
| Negative | 137 (93.2) | 154 (83.7) | 22 (61.1) | 24.468 | 4.861e−6 |
| Positive | 10 (6.8) | 30 (16.3) | 14 (38.9) | | |
| HER3 | | | | | |
| Negative | 20 (12.8) | 34 (16.5) | 7 (14.3) | 0.967 | 0.617 |
| Positive | 136 (87.2) | 172 (83.5) | 42 (85.7) | | |
| HER4 | | | | | |
| Negative | 45 (28.7) | 44 (20.5) | 11 (20.8) | 3.648 | 0.161 |
| Positive | 112 (71.3) | 171 (79.5) | 42 (79.2) | | |
| BRCA 1 | | | | | |
| Negative | 17 (10.7) | 32 (16.2) | 9 (20) | 3.411 | 0.182 |
| Positive | 142 (89.3) | 166 (83.6) | 36 (80) | | |
| P-cadherin | | | | | |
| Negative | 66 (43.1) | 82 (38.9) | 11 (21.6) | 7.584 | 0.023 |
| Positive | 87 (56.9) | 129 (61.1) | 40 (78.4) | | |
| E-cadherin | | | | | |
| Negative | 87 (43.5) | 99 (39.6) | 15 (26.8) | 5.106 | 0.078 |
| Positive | 113 (56.5) | 151 (60.4) | 41 (73.2) | | |
| EGFR | | | | | |
| Negative | 132 (83) | 175 (78.8) | 33 (61.1) | 11.452 | 0.003 |
| Positive | 27 (17) | 47 (21.2) | 21 (38.9) | | |
| CK5/6 | | | | | |
| Negative | 169 (84.1) | 213 (81.9) | 35 (61.4) | 15.219 | 0.0005 |
| Positive | 32 (15.9) | 47 (18.1) | 22 (38.6) | | |
| CK14 | | | | | |
| Negative | 174 (88.8) | 203 (81.5) | 35 (61.4) | 22.585 | 0.0001 |
| Positive | 22 (11.2) | 46 (18.5) | 22 (38.6) | | |
| P53 | | | | | |
| Negative | 160 (81.2) | 161 (64.1) | 29 (51.8) | 24.424 | 0.0005 |
| Positive | 37 (18.8) | 90 (35.9) | 27 (48.2) | | |
| Mib1 | | | | | |
| Negative | 46 (47.4) | 58 (49.6) | 3 (11.5) | 12.987 | 0.002 |
| Positive | 51 (52.6) | 59 (50.4) | 23 (88.5) | | |
| Bcl2 | | | | | |
| Negative | 7 (9.3) | 8 (8.4) | 5 (62.5) | 22.107 | 1.583e−5 |
| Positive | 68 (90.7) | 87 (91.6) | 3 (37.5) | | |

*Includes Mucoid, invasive cribriform and invasive papillary carcinoma.
**Includes ductal/NST mixed with lobular or special types.

TABLE 7
Characteristics for patients investigated for α2c
adrenoceptor protein expression.

| | Number (%) | Number (%) | Number (%) | | |
|---|---|---|---|---|---|
| | Alpha2C | Alpha2C ADR | Alph2C ADR | | |
| | ADR negative | weak positive | strong positive | | |
| Variable | expression | expression | expression | χ² | P-value |
| Patients' Age | | | | | |
| <40 | 10 (11.9) | 35 (8.8) | 4 (6.9) | 5.768 | 0.450 |
| 40-50 | 30 (35.7) | 116 (29.1) | 14 (24.1) | | |
| 51-60 | 21 (25) | 131 (32.8) | 24 (41.4) | | |
| >60 | 23 (27.4) | 117 (29.3) | 16 (27.6) | | |
| Primary tumour size | | | | | |
| ≦1.5 cm | 29 (34.5) | 133 (33.9) | 22 (39.3) | 0.622 | 0.733 |
| >1.5 cm | 55 (65.5) | 259 (66.1) | 34 (60.7) | | |
| Lymph node Stage | | | | | |
| 1 | 48 (57.1) | 235 (59.6) | 31 (55.4) | 1.778 | 0.776 |
| 2 | 30 (35.7) | 122 (31) | 21 (37.5) | | |
| 3 | 6 (7.1) | 37 (9.4) | 4 (7.1) | | |
| Grade | | | | | |
| 1 | 18 (21.4) | 66 (16.8) | 3 (5.4) | 14.090 | 0.007 |
| 2 | 34 (40.5) | 119 (30.4) | 15 (26.8) | | |
| 3 | 32 (38.1) | 207 (52.8) | 38 (67.9) | | |
| NPI | | | | | |
| Poor | 10 (11.9) | 72 (18.3) | 10 (17.5) | 6.332 | 0.176 |
| Moderate | 45 (53.6) | 218 (55.5) | 37 (64.9) | | |
| Good | 29 (34.5) | 103 26.2() | 10.(17.5) | | |
| Development of DM | | | | | |
| No | 60 (73.2) | 280 (70.4) | 40 (69) | 0.348 | 0.841 |
| Positive | 22 (26.8) | 118 (29.6) | 18 (31) | | |
| Development of | | | | | |
| Recurrence | | | | | |
| No | 49 (61.3) | 236 (60.5) | 35 (63.6) | 0.201 | 0.904 |
| Positive | 31 (38.8) | 154 (39.5) | 20 (36.4) | | |
| Vascular invasion | | | | | |
| No | 49 (58.3) | 201 (50.8) | 34 (58.6) | 4.133 | 0.388 |
| Probable | 9 (10.7) | 48 (12.1) | 9 (15.5) | | |
| Definite | 26 (31) | 147 (37.1) | 15 (25.9) | | |
| Tumour type | | | | | |
| Ductal/NST | 35 (42.7) | 244 (62.1) | 41 (70.7) | 34.183 | 0.002 |
| Lobular | 12 (14.6) | 32 (8.1) | 4 (6.9) | | |
| Tubular and | 21 (25.6) | 76 (19.3) | 6 (10.3) | | |
| Tubular mixed | | | | | |
| Medullary | 1 (1.2) | 10 (2.5) | 4 (6.9) | | |
| Other special types* | 1 (1.2) | 7 (1.8) | 0 (0) | | |
| Mixed** | 11 (13.4) | 19 (4.8) | 2 (3.4) | | |
| Menopausal status | | | | | |
| Premenopausal | 43 (51.2) | 158 (39.6) | 18 (31) | 6.276 | 0.043 |
| Postmenopausal | 41 (48.8) | 241 (60.4) | 40 (69) | | |

*Includes Mucoid, invasive cribriform and invasive papillary carcinoma.
**Includes ductal/NST mixed with lobular or special types.

TABLE 8
Associations between α2c adrenoceptor protein expression
and tumour relevant markers.

| | Number | Number | Number | | |
|---|---|---|---|---|---|
| | (%) Alpha2C | (%) | (%) | | |
| | ADR | Alpha2C | Alpha2C | | |
| | negative | ADR weak | ADR strong | | |
| Variable | expression | expression | expression | χ² | P-value |
| ER | | | | | |
| Negative | 18 (23.4) | 118 (31.3) | 19 (38.8) | 3.496 | 0.174 |
| Positive | 59 (76.6) | 259 (68.7) | 30 (61.2) | | |
| PgR | | | | | |
| Negative | 22 (29.3) | 160 (43.7) | 30 (61.2) | 12.399 | 0.002 |
| Positive | 53 (70.7) | 206 (56.3) | 19 (38.8) | | |
| AR | | | | | |
| Negative | 22 (30.1) | 126 (35.9) | 18 (38.3) | 1.092 | 0.579 |
| Positive | 51 (69.9) | 225 (64.1) | 29 (61.7) | | |
| CD71 | | | | | |
| Negative | 26 (48.1) | 64 (29.5) | 4 (14.8) | 10.817 | 0.004 |
| Positive | 28 (51.9) | 153 (70.5) | 23 (85.2) | | |
| FOXA1 | | | | | |
| Negative | 17 (41.5) | 57 (42.5) | 3 (23.1) | 1.861 | 0.394 |
| Positive | 24 (58.5) | 77 (57.5) | 10 (76.9) | | |
| CK18 | | | | | |
| Negative | 8 (11) | 64 (18.1) | 7 (14) | 2.486 | 0.289 |
| Positive | 65 (89) | 290 (81.9) | 43 (86) | | |
| HER2 | | | | | |
| Negative | 50 (65.8) | 270 (72.8) | 36 (69.2) | 1.633 | 0.442 |
| Positive | 26 (34.2) | 101 (27.2) | 16 (30.8) | | |
| HER3 | | | | | |
| Negative | 11 (17.7) | 23 (7) | 0 (0) | 12.674 | 0.002 |
| Positive | 51 (82.3) | 307 (93) | 45 (100) | | |
| HER4 | | | | | |
| Negative | 26 (40.6) | 54 (16.1) | 1 (2.3) | 30.082 | 2.935e−7 |
| Positive | 38 (59.4) | 282 (83.9) | 43 (97.7) | | |
| BRCA 1 | | | | | |
| Negative | 8 (12.9) | 51 (15.5) | 7 (17.1) | 0.385 | 0.825 |
| Positive | 54 (87.1) | 278 (84.5) | 34 (82.9) | | |
| P-cadherin | | | | | |
| Negative | 26 (42.6) | 157 (47.4) | 22 (51.2) | 0.790 | 0.674 |
| Positive | 35 (57.4) | 174 (52.6) | 21 (48.8) | | |
| E-cadherin | | | | | |
| Negative | 29 (37.7) | 127 (34) | 16 (31.4) | 0.600 | 0.741 |
| Positive | 48 (62.3) | 247 (66) | 35 (68.6) | | |
| EGFR | | | | | |
| Negative | 60 (88.2) | 265 (78.6) | 33 (70.2) | 5.740 | 0.057 |
| Positive | 8 (11.8) | 72 (21.4) | 14 (29.8) | | |
| CK5/6 | | | | | |
| Negative | 69 (89.6) | 313 (83.5) | 43 (86) | 1.933 | 0.380 |
| Positive | 8 (10.4) | 62 (16.5) | 7 (14) | | |
| CK14 | | | | | |
| Negative | 68 (94.4) | 336 (90.6) | 47 (88.7) | 1.4630 | 0.481 |
| Positive | 4 (5.5) | 35 (9.4) | 6 (11.3) | | |
| P53 | | | | | |
| Negative | 62 (82.7) | 268 (71.8) | 34 (65.4) | 5.301 | 0.071 |
| Positive | 13 (17.3) | 105 (28.2) | 18 (34.6) | | |
| Mib1 | | | | | |
| Negative | 20 (62.5) | 89 (46.1) | 8 (30.8) | 5.888 | 0.053 |
| Positive | 12 (37.5) | 104 (53.9) | 18 (69.2) | | |
| Bcl2 | | | | | |
| Negative | 5 (11.9) | 20 (10.6) | 4 (22.2) | 2.167 | 0.338 |
| Positive | 37 (88.1) | 169 (89.4) | 14 (77.8) | | |

*Includes Mucoid, invasive cribriform and invasive papillary carcinoma.
**Includes ductal/NST mixed with lobular or special types.

TABLE 9
Characteristics for patients investigated for β2
adrenoceptor protein expression.

| | Number (%) | Number (%) | Number (%) | | |
|---|---|---|---|---|---|
| | B2ADR | B2 ADR | B2 ADR | | |
| | negative | weak positive | strong positive | | |
| Variable | expression | expression | expression | χ² | P-value |
| Patients' Age | | | | | |
| <40 | 3 (17.6) | 31 (9.8) | 14 (3.3) | 18.332 | 0.005 |
| 40-50 | 4 (23.5) | 93 (29.5) | 122 (28.7) | | |
| 51-60 | 6 (35.3) | 92 (29.2) | 148 (34.8) | | |
| >60 | 4 (23.5) | 99 (31.4) | 141 (33.2) | | |
| Primary tumour size | | | | | |
| ≦1.5 cm | 9 (52.9) | 92 (29.3) | 165 (39) | 9.815 | 0.007 |
| >1.5 cm | 8 (47.1) | 222 (70.7) | 258 (61) | | |
| Lymph node Stage | | | | | |
| 1 | 14 (87.5) | 212 (67.7) | 266 (62.6) | 12.129 | 0.016 |
| 2 | 0 (0) | 65 (20.8) | 122 (28.7) | | |
| 3 | 2 (12.5) | 36 (11.5) | 37 (8.7) | | |
| Grade | | | | | |
| 1 | 0 (0 | 35 (11.1) | 123 (29.1) | 58.470 | 6.081e−12 |
| 2 | 6 (35.3) | 98 (31.2) | 158 (37.4) | | |
| 3 | 11 (64.7) | 181 (57.6) | 142 (33.6) | | |
| NPI | | | | | |
| Poor | 2 (11.8) | 49 (15.6) | 53 (12.5) | 28.020 | 1.235e−5 |
| Moderate | 12 (70.6) | 194 (61.8) | 200 (47.2) | | |
| Good | 3 (17.6) | 71 (22.6) | 171 (40.3) | | |
| Development of DM | | | | | |
| No | 10 (58.8) | 217 (69.6) | 273 (64.4) | 2.596 | 0.273 |
| Positive | 7 (41.2) | 95 (30.4) | 151 (35.6) | | |
| Development of DM | | | | | |
| Recurrence | | | | | |
| No | 7 (43.8) | 177 (57.3) | 221 (52.4) | 3.151 | 0.533 |
| Positive | 9 (56.3) | 132 (42.7) | 200 (47.4) | | |
| Vascular invasion | | | | | |
| No | 9 (56.3) | 182 (58.1) | 254 (60.2) | 7.023 | 0.135 |
| Probable | 4 (25) | 27 (8.6) | 49 (11.6) | | |
| Definite | 3 (18.8) | 104 (33.2) | 119 (28.2) | | |
| Tumour type | | | | | |
| Ductal/NST | 9 (56.3) | 206 (66.7) | 186 (44.4) | | |
| Lobular | 1 (6.3) | 30 (9.7) | 57 (13.6) | | |
| Tubular and | 1 (6.3) | 43 (13.9) | 130 (31) | | |
| Tubular mixed | | | | | |
| Medullary | 1 (6.3) | 14 (4.5) | 6 (1.4) | 65.800 | 1.101e−8 |
| Other special types* | 0 (0) | 7 (2.3) | 8 (1.9) | | |
| Mixed** | 3 (18.8) | 7 (2.3) | 29 (6.9) | | |
| Menopausal status | | | | | |
| Premenopausal | 7 (41.2) | 128 (40.6) | 143 (33.6) | 3.950 | 0.139 |
| Postmenopausal | 10 (58.8) | 187 (59.4) | 282 (66.4) | | |

*Includes Mucoid, invasive cribriform and invasive papillary carcinoma.
**Includes ductal/NST mixed with lobular or special types.

TABLE 10
Associations between β2 adrenoceptor protein expression
and tumour relevant markers.

| | Number (%) | Number (%) | Number (%) | | |
|---|---|---|---|---|---|
| | B2 ADR | B2 ADR | B2 ADR | | |
| | negative | weak | strong | | |
| Variable | expression | expression | expression | χ² | P-value |
| ER | | | | | |
| Negative | 9 (64.3) | 131 (44.7) | 78 (20.4) | 52.588 | 3.807e−12 |
| Positive | 5 (35.7) | 162 (55.3) | 305 (79.6) | | |
| PgR | | | | | |
| Negative | 9 (56.3) | 174 (60.2) | 131 (34.5) | 44.502 | 2.170e−10 |
| Positive | 7 (43.8) | 115 (39.8) | 249 (65.5) | | |
| AR | | | | | |
| Negative | 9 (56.3) | 147 (54) | 106 (30.1) | 37.932 | 5.795e−9 |
| Positive | 7 (43.8) | 125 (46) | 246 (69.9) | | |
| CD71 | | | | | |
| Negative | 9 (64.3) | 83 (33.3) | 110 (34.5) | 5.619 | 0.060 |
| Positive | 5 (35.7) | 166 (66.7) | 209 (65.5) | | |
| FOXA1 | | | | | |
| Negative | 6 (46.2) | 110 (52.9) | 123 (41.6) | 6.309 | 0.043 |
| Positive | 7 (53.8) | 98 (47.1) | 173 (58.4) | | |
| CK18 | | | | | |
| Negative | 6 (50) | 72 (27.6) | 11 (3.1) | 86.243 | 1.872e−19 |
| Positive | 6 (50) | 189 (72.4) | 341 (96.9) | | |
| HER2 | | | | | |
| Negative | 11 (73.3) | 184 (83.3) | 248 (83.5) | 1.058 | 0.589 |
| Positive | 4 (26.7) | 37 (16.7) | 49 (16.5) | | |
| HER3 | | | | | |
| Negative | 0 (0) | 29 (12.3) | 56 (18.4) | 5.427 | 0.066 |
| Positive | 9 (100) | 207 (87.7) | 249 (81.6) | | |
| HER4 | | | | | |
| Negative | 1 1(0) | 48 (20.6) | 90 (28.7) | 5.858 | 0.053 |
| Positive | 9 (90) | 185 (79.4) | 224 (71.3) | | |
| BRCA 1 | | | | | |
| Negative | 3 (33.3) | 43(18.8) | 28 (9.1) | 13.574 | 0.001 |
| Positive | 6 (66.7) | 186 (81.2) | 280 (90.9) | | |
| P-cadherin | | | | | |
| Negative | 3 (33.3) | 75 (32.1) | 147 (48.8) | 15.538 | 0.0004 |
| Positive | 6 (66.7) | 159 (67.9) | 154 (51.2) | | |
| E-cadherin | | | | | |
| Negative | 4 (26.7) | 135 (46.1) | 173 (44.9) | 2.174 | 0.337 |
| Positive | 11 (73.3) | 158 (53.9) | 212 (55.1) | | |
| EGFR | | | | | |
| Negative | 6 (66.7) | 188 (76.7) | 264 (82.5) | 3.837 | 0.147 |
| Positive | 3 (33.3) | 57 (23.3) | 56 (17.5) | | |
| CK5/6 | | | | | |
| Negative | 11 (68.8) | 226 (74.3) | 345 (85.6) | 15.448 | 0.0004 |
| Positive | 5 (31.3) | 78 (25.7) | 58 (14.4) | | |
| CK14 | | | | | |
| Negative | 11 (78.6) | 238 (80.4) | 332 (85.3) | 3.136 | 0.208 |
| Positive | 3 (21.4) | 58 (19.6) | 57 (14.7) | | |
| P53 | | | | | |
| Negative | 10 (71.4) | 194 (66.7) | 300 (77.1) | 9.161 | 0.010 |
| Positive | 4 (28.6) | 97 (33.3) | 89 (22.9) | | |
| Mib1 | | | | | |
| Negative | 6 (54.5) | 60 (38) | 112 (55.4) | 11.038 | 0.004 |
| Positive | 5 (45.5) | 98 (62) | 90 (44.6) | | |
| Bcl2 | | | | | |
| Negative | 0 (0) | 17 (16.8) | 10 (6) | 8.863 | 0.012 |
| Positive | 5 (100) | 84 (83.2) | 157 (94) | | |

*Includes Mucoid, invasive cribriform and invasive papillary carcinoma.
**Includes ductal/NST mixed with lobular or special types.

1. A method for the prevention of cancer metastasis or tumour recurrence in a patient comprising administering an adrenergic receptor antagonist to a patient in need thereof.

2. (canceled)

3. A method as claimed in claim 1, in which the metastasis or tumour is associated with breast cancer, colon cancer, prostate cancer or ovarian cancer.

4. A method as claimed in claim 1, in which the cancer is basal-like breast cancer.

5. A method as claimed in claim 1, in which the adrenergic receptor antagonist is administered with another pharmaceutically active agent.

6. A method as claimed in claim 1, in which the adrenergic receptor antagonist is a beta-adrenergic receptor antagonist.

7. A method as claimed in claim 6, in which the beta-adrenergic receptor antagonist is selected from the group consisting of Acebutolol, Alprenolol, Atenolol, Bisoprolol, Bucinodolol, Carteolol, Carvedilol, Celiprolol, Labetalol, Metoprolol, Nadolol, Nebivolol, Oxyprenolol, Penbutalol, Pindolol, Propranolol, Sotalol, and Timolol.

8. A method as claimed in claim 1, in which the adrenergic receptor antagonist is an alpha-adrenergic receptor antagonist.

9. A method as claimed in claim 8, in which the alpha-adrenergic receptor antagonist is selected from the group consisting of Alfuzosin, Doxazosin, Ergotamine, Indoramin, Prazosin, Phenoxybenzamine, Phentolamine, Tamsulosin, Terazosin, Tolazoline, and Trimazosin.

10-11. (canceled)

12. A kit of parts for use in a method as defined in claim 1, comprising an adrenergic receptor antagonist, and an administration vehicle.

13. A method for identifying a patient at risk of developing cancer comprising the step of assaying for levels of expression of adrenergic receptor proteins in said patient.

14. A method as claimed in claim 13, in which the adrenergic receptor protein is an alpha adrenergic receptor protein.

15. A method as claimed in claim 13, in which the adrenergic receptor protein is a beta adrenergic receptor protein.
